(19) United States
(12) Patent Application Publication
(10) Pub. No.: US 2003/0143167 A1
(43) Pub. Date: Jul. 31, 2003

(54) Vitamin B6 derivatives as protective components in the oxidative treatment of hair

(76) Inventor: Astrid Kleen, Erkrath (DE)

Correspondence Address: HENKEL CORPORATION, 2500 RENAISSANCE BLVD, STE 200, GULPH MILLS, PA, 19406, US

(21) Appl. No.: 10/311,188

(22) PCT Filed: Jun. 9, 2001
(86) PCT No.: PCT/EP01/06547

(30) Foreign Application Priority Data
Jun. 20, 2000 (DE) 100 30 313.7
Apr. 26, 2001 (DE) 101 20 305.5

(51) Int. Cl. A61K 7/135

(52) U.S. Cl. 424/62

(57) ABSTRACT

The invention relates to the use of vitamin B6 derivatives, preferably pyridoxine, pyridoxal, pyridoxal-5′-phosphate and pyridoxamine for prevention of damage to keratin fibres.

This invention relates to the use of vitamin B6 derivatives for reducing the damage to keratinous fibers, especially human hair, by oxidative processes. The invention also relates to preparations for the oxidative treatment of hair, more particularly for the blonding of hair, which contain vitamin B6 derivatives as structure-stabilizing component and to a process for the oxidative treatment of keratin fibers.

Keratin fibers in the context of the present invention are understood to include pelts, wool, feathers and, in particular, human hair. Nowadays, human hair is treated in many different ways with hair-care preparations. Such treatments include, for example, the cleaning of hair with shampoos, the care and regeneration of hair with rinses and conditioners and the bleaching, coloring and shaping of hair with coloring and tinting formulations, wave formulations and styling preparations. The oxidative treatment of hair is an essential step in the permanent coloring of hair with oxidation colorants, in the blonding of hair and in the permanent shaping of hair.

The blonding or decoloring of hair is achieved by oxidation of the natural hair pigments or by oxidation of the artificial hair dye. The basic principles of blonding processes are known to the expert and are comprehensively described in relevant monographs, for example by K. Schrader in “Grundlagen und Rezepturen der Kosmetika”, 2nd Edition, 1989, Dr. Alfred Hüthig Verlag, Heidelberg, and by W. Umbach (Ed.) in “Kosmetik”, 2nd Edition, 1995, Georg Thieme Verlag, Stuttgart/New York. Besides their desired decoloring effect, blonding preparations can damage the structure of hair keratin, particularly if they are used frequently. The hair is in danger of more serious damage when it is exposed to the effect of so-called booster components, such as ammonium peroxodisulfate for example, which can be dissolved in relatively high concentrations in the final preparations. Such damage is reflected in increasing fragility, poor combability and a deterioration in the hold and body of the hair. In addition, structurally damaged hair is often dull and lackluster in appearance. These problems should be tackled with structure-improving additives, for example in the blonding process in which the additives are advantageously a constituent of the blonding preparation itself.

For these reasons, there is a need for new hair-care agents which would enable the parameters mentioned to be improved and which would be compatible with oxidizing hair treatment preparations.

Pyridoxine and other compounds belonging to the vitamin B6 group have already been proposed for use as components in hair tonics for reducing refatting and for stimulating hair growth. EP 0678293 A2 proposes topical compositions containing pyridoxine tripropionate for treating the hair and skin. EP 001079 A1 describes anti-seborrheic cosmetic compositions containing pyridoxine tripalmitate as their active ingredient. Derivatives of pyridoxine, pyridoxal or pyridoxamine as active ingredients of oxidizing hair treatment preparations have not hitherto been known to the expert.

It has surprisingly been found that pyridoxine, pyridoxal or pyridoxamine derivatives eminently satisfy the requirements stated above. Vitamin B6 and certain derivatives improve the structure of hair keratin, even during the oxidative hair treatment process, without affecting the lightening or permanent waving result.

In a first embodiment, therefore, the present invention relates to the use of compounds corresponding to formula (I):

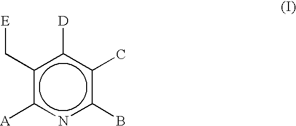

in which

A and B independently of one another represent hydrogen, halogen, a C₁₋₄ alkyl group, a C₃₋₆ cycloalkyl group, a C₁₋₄ monohydroxyalkyl group, a C₂₋₄ oligohydroxyalkyl group, a C₁₋₄ aminoalkyl group, a group —OR or a group —NR¹R², where R¹ and R² independently of one another represent hydrogen, a C₁₋₄ alkyl group or a C₁₋₄ monohydroxyalkyl group or R¹ and R² together with the nitrogen atom form a saturated ring,

C represents a group —OR, —NR¹R², —OP(O)(0R³)₂, a C₁₋₄ monohydroxyalkyl group, a C₂₋₄ oligohydroxyalkyl group or a C₁₋₄ alkyl group,

D represents a hydroxy group, a carboxy group, a C₁₋₂₂ alkoxy-carbonyl group, a formyl group, a group —CH₂OR or a group —CH₂—NR₂,

E represents a group —OR, —OP(O)(OR³)₂, a C₁₋₄ monohydroxyalkyl group or a C₂₋₄ oligohydroxyalkyl group,

R representing hydrogen, a C₁₋₄ alkyl group, a C₁₋₂₂ acyl group, a hydroxy-C₂₋₂₂-acyl group, a C₂₋₁₀ carboxyacyl group, a C₃₋₁₀ oligocarboxyacyl group, an oligocarboxymonohydroxy-C₃₋₁₀-acyl group, an oligocarboxyoligohydroxy-C₃₋₁₀-acyl group, a C₃₋₈ cycloalkyl group, a C₁₋₄ monohydroxyalkyl group, a C₂₋₄ oligohydroxyalkyl group, an aryl group which may contain a hydroxy, nitro or amino group, a heteroaromatic group or a group —CH₂CH₂NR¹R², where R¹ and R² are as defined above,

R³ representing hydrogen or a C₁₋₅ alkyl group,

or one of the corresponding physiologically compatible salts, for reducing the damage to keratinous fibers, especially human hair, by oxidative processes. The corresponding preparations containing these vitamin B6 derivatives are intended for topical application.

Examples of C₁₋₄ alkyl groups in the compounds according to the invention are methyl, ethyl, n-propyl, isopropyl, n-butyl and tert.butyl. Preferred alkyl groups are methyl and ethyl. Methyl is a particularly preferred alkyl group. Preferred C₃₋₆ cycloalkyl groups are cyclopropyl, cyclobutyl, cyclopentyl, cyclohexyl. Cyclohexyl and cyclopentyl are particularly preferred groups. Preferred C₁₋₄ monohydroxyalkyl groups are the groups hydroxymethyl, 2-hydroxyethyl, 3-hydroxypropyl or 4-hydroxybutyl; hydroxymethyl and 2-hydroxyethyl are particularly preferred monohydroxyalkyl groups. A preferred C₂₋₄ oligohydroxyalkyl group is the 1,2-dihydroxyethyl group. Preferred C₁₋₂₂ acyl groups are, for example, acetyl, propionyl, butyryl, valeryl, capryl, lauryl, myristyl, palmityl, stearyl, linolyl, behenyl. Examples of a hydroxy-C₂₋₂₂-acyl group are salicylic acid or lactic acid. Preferred C₂₋₁₀ carboxyacyl groups are derived, for example, from malonic acid, succinic acid or adipic acid. One example of a preferred C₃₋₁₀ oligocarboxyacyl group is glyceric acid. A preferred oligocarboxymonohydroxy-C₃₋₁₀-acyl group is derived, for example, from citric acid or malic acid. Preferred oligocarboxyoligohydroxy-C₃₋₁₀-acyl groups are derived, for example, from tartaric acid. According to the invention, preferred halogen substituents are fluorine, chlorine, bromine and iodine; chlorine and bromine are particularly preferred. Physiologically compatible salts in the context of the invention are salts of inorganic or organic acids, for example hydrochlorides, sulfates or hydrobromides. According to the invention, the other terms used are derived from the definitions given here.

The ester derivatives of the compounds corresponding to formula (I) also have physiological and hair-structure-improving properties. This applies in particular to the esters of pyridoxine which can be converted by hydrolysis into pyridoxine. In addition, the ester derivatives acquire improved lipid solubility compared with the non-esterified derivatives. Other examples of carboxylic acid ester derivatives of pyridoxine are derived from the carboxylic acids, such as formic acid, acetic acid, propionic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, suberic acid, azelaic acid, sebacic acid, propiolic acid, crotonic acid, isocrotonic acid, elaidic acid, maleic acid, fumaric acid, muconic acid, citraconic acid, mesaconic acid, camphor acid, benzoic acid, o,m,p-phthalic acid, naphthoic acid, toluylic acid, hydratropic acid, atropic acid, cinnamic acid, isonicotinic acid, nicotinic acid, bicarbamic acid, 4,4′-dicyano-6,6′-binicotinic acid, 8-carbamoyloctanoic acid, 1,2,4-pentanetricarboxylic acid, 2-pyrrole carboxylic acid, 1,2,4,6,7-naphthalene pentaacetic acid, malonaldehydic acid, 4-hydroxyphthalamidic acid, 1-pyrazole carboxylic acid, gallic acid or propane tricarboxylic acid, and from dicarboxylic acids selected from the group consisting of compounds corresponding to general formula (II):

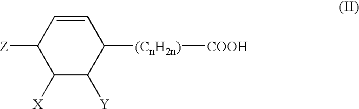

in which Z represents a linear or branched alkyl or alkenyl group containing 4 to 12 carbon atoms, n is a number of 4 to 12 and one of the two groups X and Y represents a COOH group and the other represents hydrogen or a methyl or ethyl group, dicarboxylic acids corresponding to general formula (II) which additionally contain 1 to 3 methyl or ethyl substituents on the cyclohexene ring and dicarboxylic acids which formally are formed from the dicarboxylic acids (II) by addition of one molecule of water onto the double bond in the cyclohexene ring.

Compounds corresponding to formula (I) in which one of the two groups A and B is hydrogen are preferred.

Compounds corresponding to formula (I) in which one of the two groups A and B is hydrogen and the other group is a C₁₋₄ alkyl group are preferred.

Other preferred compounds of formula (I) are those in which C is a hydroxy group, a C₁₋₄ monohydroxyalkyl group or a C₂₋₄ oligohydroxyalkyl group.

According to the invention, compounds of formula (I) in which D is a hydroxymethyl group, a group —CH₂—NR₂, a hydroxy group, a carboxy group or a formyl group are preferred.

Other preferred compounds of formula (I) are those in which E is a hydroxy group or a group —OP(O)(OH)₂.

A particularly preferred compound corresponding to formula I is selected from pyridoxine, pyridoxal, pyridoxamine and pyridoxal-5′-phoshate.

Compounds corresponding to formula (I) such as, for example, pyridoxal, pyridoxal-5-phosphonic acid, pyridoxamine, pyridoxine and 3-hydroxy-5-hydroxymethyl-2-methylpyridine-4-carboxylic acid are commercially obtainable.

In a second embodiment, the present invention relates to a preparation for blonding keratin fibers, especially human hair, containing at least one oxidizing agent, characterized in that the preparation contains at least one compound corresponding to formula (I) or one of the corresponding physiologically compatible salts, preferably in a quantity of 0.05 to 2% by weight, based on the preparation.

The preparations according to the invention preferably contain hydrogen peroxide or an addition compound of hydrogen peroxide onto inorganic or organic compounds such as, for example, sodium perborate, sodium percarbonate, sodium percarbamide, polyvinyl pyrrolidone, urea peroxide and melamine peroxide as oxidizing agents.

The blonding effect may advantageously be enhanced by so-called “boosters”. These are generally solid peroxo compounds which are not addition products of hydrogen peroxide onto other components. Basically, the choice of these peroxo compounds is not subject to any restrictions. Standard peroxo compounds known to the expert are, for example, ammonium peroxodisulfate, potassium peroxodisulfate, sodium peroxodisulfate, ammonium persulfate, potassium persulfate, sodium persulfate, potassium peroxodiphosphate, percarbonates, such as magnesium percarbonate, and peroxides, such as barium peroxide. Of these peroxo compounds, which may also be used in combination, the inorganic compounds are preferred for the purposes of the invention. The peroxodisulfates, especially ammonium peroxodisulfate, are particularly preferred.

The peroxo compounds are present in the blonding preparations according to the invention in quantities of preferably 1 to 30% by weight and more particularly 5 to 20% by weight.

The blonding preparations according to the invention contain an alkalizing agent to establish the alkaline pH of the mixture applied as another important component. According to the invention, the usual alkalizing agents known to the expert for blonding preparations, such as ammonium, alkali metal and alkaline earth metal hydroxides, carbonates, hydrogen carbonates, hydroxycarbonates, silicates, more particularly metasilicates, and alkali metal phosphates, may be used. In one preferred embodiment, the blonding preparations according to the invention contain at least two different alkalizing agents. Mixtures of, for example, a metasilicate and a hydroxycarbonate can be preferred.

The preparations contain alkalizing agents in quantities of preferably 1 to 25% by weight and more preferably 1.5 to 20% by weight.

The preparation according to the invention may additionally contain another care component as an active ingredient capable of stabilizing the hair structure.

Examples of preferred structure-improving agents according to the invention are vitamins and derivatives or precursors thereof. According to the invention, panthenol and its physiologically compatible derivatives are particularly preferred. Such derivatives are in particular the esters and ethers of panthenol and cationically derivatized panthenols. Individual representatives are, for example, panthenol triacetate, panthenol monoethylether and its monoacetate and the cationic panthenol derivatives disclosed in WO 92/13829 A1. According to the invention, another preferred panthenol derivative is its precursor pantolactone. Within this group, panthenol is preferred.

Polyvinyl pyrrolidone (PVP) is also known for its fiber-restructuring properties and is preferred for the purposes of the invention.

According to the invention, other particularly effective restructuring compounds are the aldehydes. Particularly preferred examples are formaldehyde and formaldehyde donors, such as for example methoxymethyl ester, dimethylol (thio)urea derivatives, oxazolidine derivatives, N-hydroxymethyl maleic imide, hexamethylene tetramine and derivatives thereof, hydantoin derivatives, pyridinium-substituted dimethyl ethers, imidazolidinyl urea derivatives, isothiazolinones, 2-bromo-2-nitropropanediol and 5-bromo-5-nitro-1,3-dioxane. Other particularly preferred aldehydes are acetaldehyde, glyoxal, glycerinaldehyde and glutardialdehyde.

Another suitable group of restructuring agents are plant extracts.

These extracts are normally prepared by extraction of the whole plant. However, it may be preferred in individual cases to prepare the extracts exclusively from blossoms and/or leaves of the plant.

So far as the plant extracts suitable for use in accordance with the invention are concerned, particular reference is made to the extracts listed in the Table beginning on page 44 of the 3rd Edition of the Leitfaden zur lnhaltsstoffdeklaration kosmetischer Mittel, published by the Industrieverband Körperpflege-und Waschmittel e.V. (IKW), Frankfurt.

According to the invention, preference is attributed above all to the extracts of oak bark, stinging nettle, hamamelis, hops, chamomile, burdock root, horse willow, hawthorn, lime blossom, almond, aloe vera, pine needle, horse chestnut, sandalwood, juniper, coconut, mango, apricot, lemon, wheat, kiwi, melon, orange, grapefruit, sage, rosemary, birch, mallow, lady's smock, creeping thyme, yarrow, thyme, balm, restharrow, coltsfoot, hibiscus, meristem, green tea, ginseng and ginger root.

Particularly preferred extracts are those of oak bark, stinging nettle, hamamelis, hops, camomile, burdock root, horse willow, lime blossom, almond, aloe vera, coconut, mango, apricot, lemon, wheat, kiwi, melon, orange, grapefruit, sage, rosemary, birch, lady's smock, creeping thyme, yarrow, restharrow, meristem, green tea, ginseng and ginger root.

Extracts of almond, aloe vera, coconut, mango, apricot, lemon, wheat, kiwi, melon and green tea are most particularly suitable for the preparations according to the invention.

Extractants suitable for the preparation of the plant extracts mentioned include water, alcohols and mixtures thereof. Among the alcohols, lower alcohols, such as ethanol and isopropanol, but especially polyhydric alcohols, such as ethylene glycol and propylene glycol, both as sole extractant and in admixture with water are preferred. Plant extracts based on water/propylene glycol in a ratio of 1:10 to 10:1 have proved to be particularly suitable.

According to the invention, the plant extracts may be used both in pure form and in dilute form. If they are used in dilute form, they normally contain ca. 2 to 80% by weight active substance and—as solvent—the extractant or mixture of extractants used in their preparation.

In another preferred embodiment, mixtures of several, more particularly two, different plant extracts are used in the preparations according to the invention.

According to the invention, other preferred restructuring agents are honey extracts. These extracts are obtained similarly to the plant extracts and typically contain 1 to 10% by weight and more particularly 3 to 5% by weight of active substance. Water/propylene glycol mixtures may again be preferred extractants.

Other restructuring agents are protein hydrolyzates, more particularly elastin, collagen, keratin, milk protein, soya protein, almond protein and wheat protein hydrolyzates, condensation products thereof with fatty acids and quaternized protein hydrolyzates. Highly degraded keratin hydrolyzates with molecular weights in the range from 400 to 800 are particularly preferred for the purposes of the invention, as are quaternized protein hydrolyzates such as those marketed, for example, under the names of Gluadin® WQ (INCI name: Laurdimonium Hydroxypropyl Hydrolyzed Wheat Protein) and Crotein® Q (INCI name: Hydroxypropyltrimonium Hydrolyzed Collagen).

Besides the quaternized protein hydrolyzates, quaternary polymers are also preferred restructuring compounds for the purposes of the invention. The polymers marketed under the names of Mirapol® A15 (INCI name: Polyquaternium-2), Onamer® M (INCI name: Polyquaternium-1) and Merquat® 100 (INCI name: Polyquaternium-6) are particularly preferred.

Other fiber-restructuring agents are mono-, di- and oligosaccharides such as, for example, glucose, galactose, fructose, sucrose and lactose. Derivatives of these pentoses and hexoses, such as the corresponding onic and uronic acids (sugar acids), sugar alcohols, sugar amines such as, for example, N-glucosamine and glycosides may also be used in accordance with the invention. According to the invention, the sugar acids may be used in free form, in the form of their salts, preferably calcium, magnesium and zinc salts, and in the form of their esters or lactones. Preferred sugar acids are gluconic acid, gluconic acid-γ-lactone, lactobionic acid, glucuronic acid and mono- or dilactones thereof, pangamic acid, saccharic acid, mannosaccharic acid and mono- or dilactones thereof and mucic acid and mono- or dilactones thereof. Preferred sugar alcohols are sorbitol, mannitol and dulcitol. Preferred glycosides are the methyl glucosides. Within this group, glucose, N-glucosamine and gluconic acid are particularly preferred.

Certain amino acids may also be used as hair-restructuring agents in accordance with the invention. Examples are the amino acids serine, threonine and tyrosine described in DE 195 22 569 to which reference is specifically made here. Derivatives of serine, for example serine phosphate, are also preferred for the purposes of the invention. Other restructuring amino acids are iysine and arginine. Serine and arginine are particularly preferred fiber-restructuring agents.

Certain acids, more particularly α-hydroxycarboxylic acids, and salts thereof may also be used for restructuring. According to the invention, preferred restructuring acids are lactic acid, malic acid, tartaric acid, glyceric acid and maleic acid. Lactic acid is particularly preferred. In addition, special phosphonic acids and their salts improve the structure of keratinous fibers. According to the invention, preferred phosphonic acids are n-octylphosphonic acid and n-decylphosphonic acid.

Lipid-soluble ester alcohols or ester polyols are also known for their restructuring effect. They may be regarded as lipid-soluble when 5% by weight of these products dissolve clearly in cetyl alcohol at 80° C.

The ester alcohols or ester polyols suitable for the purposes of the invention are obtainable by reaction of an epoxyfatty acid ester with water or mono- or polyhydric C₁₋₁₀ alcohols with opening of the epoxide ring and formation of a vicinal dihydroxyethyl or hydroxyalkoxy ethyl group. The epoxyfatty acid ester may also be an epoxidation product of a technical fatty acid ester containing some saturated fatty acids. However, the epoxide oxygen content should amount to at least 3% by weight and preferably to between 5 and 10% by weight.

The epoxy fatty acid esters are either epoxidized fatty acid esters of monohydric alcohols, i.e. for example epoxidized oleic acid methyl ester, linoleic acid methyl ester, ricinoleic acid methyl ester or epoxidized fatty acid esters of polyhydric alcohols, for example glycerol monooleate or propylene glycol monooleate or epoxidized fatty acid triglycerides, for example oleic acid triglyceride, or unsaturated oils such as, for example, olive oil, soybean oil, sunflower oil, linseed oil, rapeseed oil.

Of particular technical interest are, above all, unsaturated fatty acid methyl ester epoxides of unsaturated vegetable fatty acids. The reaction product of a vegetable oil fatty acid methyl ester epoxidate with a C₂₋₆ polyol containing 2 to 6 hydroxyl groups is a particularly preferred ester polyol. Ethylene glycol, 1,2-propylene glycol, 1,3-propylene glycol, butanediol, pentanediol, hexanediol, glycerol, trimethylol propane, pentaerythritol, sorbitol or diglycerol, for example, may be present as polyols.

The reaction product of a vegetable fatty acid methyl ester epoxidate with trimethylol propane having a hydroxyl value of 350 to 450 is a particularly suitable ester polyol for the hair treatment preparations according to the invention. One such product based on soybean oil fatty acid methyl ester epoxide and trimethylol propane is obtainable under the name of Sovermol®760.

Vitamin B₃ may also be used as a restructuring agent. The compounds nicotinic acid and nicotinic acid amide (niacin amide) often go under that name. Nicotinic acid amide is preferred for the purposes of the invention.

Vitamin H may also be used as a restructuring agent in accordance with the invention. Vitamin H is the name for (3aS,4S,6aR)-2-oxohexahydrothienol[3,4-d]-imidazole-4-valeric acid which is now known by the trivial name of biotin.

According to the invention, particularly preferred structure-improving agents are selected from panthenol, physiologically compatible panthenol derivatives, mono-, di- and oligosaccharides, serine, arginine, niacinamide, polyvinyl pyrrolidone, gluconic acid, biotin and the lipid-soluble ester alcohols or ester polyols.

The preparations according to the invention contain the additional restructuring agents preferably in quantities of 0.1 to 5% by weight and more particularly in quantities of 0.2 to 2% by weight.

The preparations according to the invention may be present as solids or may be incorporated in a suitable aqueous, alcoholic or aqueous/alcoholic carrier. For blonding purposes, such carriers are, for example, creams, emulsions, gels, pastes or other preparations suitable for application to the hair. Aqueous/alcoholic solutions in the context of the present invention are aqueous solutions containing 3 to 70% by weight of a C₁₋₄ alcohol, more particularly ethanol or isopropanol. The preparations according to the invention may additionally contain other organic solvents such as, for example, methoxybutanol, benzyl alcohol, ethyl diglycol or 1,2-propylene glycol. Water-soluble organic solvents are preferred.

For blonding human hair, more particularly for tress application, solid or paste-form preparations containing solid oxidizing agents are normally mixed with a dilute hydrogen peroxide solution immediately before application. This mixture is then applied to the hair and rinsed out again after a certain contact time. The concentration of this hydrogen peroxide solution is determined on the one hand by legal requirements and, on the other hand, by the desired effect. 6% to 12% solutions in water are generally used. The quantity ratios of blonding preparation and hydrogen peroxide solution are normally in the range from 1:1 to 1:2, an excess of hydrogen peroxide solution being selected in particular when a pronounced blonding effect is required.

If the preparation is made up as a powder, it generally contains an additional component for dedusting the finely powdered formulation. Such dedusting agents are typically oils, liquid waxes, ether derivatives. International patent application WO 00/30596 discloses solvents liquid at 25° C. selected from the group of hydrocarbons, alcohols, esters and ketones such as, for example, 3-methoxybutanol, benzyl alcohol, propane-1,2-diol, hexanol, cyclohexanone, propylene carbonate and ethyl diglycol which are suitable for dedusting and which may be present in the preparations according to the invention.

The preparations according to the invention may also contain any of the known active substances, additives and auxiliaries typical of such preparations. In many cases, the preparations contain at least one surfactant, both anionic and zwitterionic, ampholytic, nonionic and cationic surfactants being suitable in principle. In many cases, however, it has been found to be of advantage to select the surfactants from anionic, zwitterionic or nonionic surfactants.

Suitable anionic surfactants for the preparations according to the invention are any anionic surface-active substances suitable for use on the human body. Such substances are characterized by a water-solubilizing anionic group such as, for example, a carboxylate, sulfate, sulfonate or phosphate group and a lipophilic alkyl group containing around 10 to 22 carbon atoms. In addition, glycol or polyglycol ether groups, ester, ether and amide and hydroxyl groups may also be present in the molecule. The following are examples of suitable anionic surfactants—in the form of the sodium, potassium and ammonium salts and the mono-, di- and trialkanol-ammonium ammonium salts containing 2 or 3 carbon atoms in the alkanol group:

linear fatty acids containing 10 to 22 carbon atoms (soaps),

ether carboxylic acids corresponding to the formula R—O—(CH₂—CH₂P)ₓ—CH₂—COOH, in which R is a linear alkyl group containing 10 to 22 carbon atoms and x=0 or 1 to 16,

acyl sarcosides containing 10 to 18 carbon atoms in the acyl group,

acyl taurides containing 10 to 18 carbon atoms in the acyl group,

acyl isethionates containing 10 to 18 carbon atoms in the acyl group,

sulfosuccinic acid mono- and dialkyl esters containing 8 to 18 carbon atoms in the alkyl group and sulfosuccinic acid monoalkyl polyoxyethyl esters containing 8 to 18 carbon atoms in the alkyl group and 1 to 6 oxyethyl groups,

linear alkane sulfonates containing 12 to 18 carbon atoms,

linear α-olefin sulfonates containing 12 to 18 carbon atoms,

α-sulfofatty acid methyl esters of fatty acids containing 12 to 18 carbon atoms,

alkyl sulfates and alkyl polyglycol ether sulfates corresponding to the formula R—O(CH₂—CH₂O)ₓ—SO₃H, in which R is a preferably linear alkyl group containing 10 to 18 carbon atoms and x=0 or 1 to 12,

mixtures of surface-active hydroxysulfonates according to DE-A-37 25 030,

sulfated hydroxyalkyl polyethylene and/or hydroxyalkylene propylene glycol ethers according to DE-A-37 23 354,

sulfonates of unsaturated fatty acids containing 12 to 24 carbon atoms and 1 to 6 double bonds according to DE-A-39 26 344,

esters of tartaric acid and citric acid with alcohols in the form of addition products of around 2 to 15 molecules of ethylene oxide and/or propylene oxide with fatty alcohols containing 8 to 22 carbon atoms.

Preferred anionic surfactants are alkyl sulfates, alkyl polyglycol ether sulfates and ether carboxylic acids containing 10 to 18 carbon atoms in the alkyl group and up to 12 glycol ether groups in the molecule and, in particular, salts of saturated and, more particularly, unsaturated C₈₋₂₂ carboxylic acids, such as oleic acid, stearic acid, isostearic acid and palmitic acid.

In the context of the invention, zwitterionic surfactants are surface-active compounds which contain at least one quaternary ammonium group and at least one —COO⁽⁻⁾ or —SO₃⁽⁻⁾ group in the molecule. Particularly suitable zwitterionic surfactants are the so-called betaines, such as N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. A preferred zwitterionic surfactant is the fatty acid amide derivative known by the INCI name of Cocamidopropyl Betaine.

Ampholytic surfactants are surface-active compounds which, in addition to a C₈₋₁₈ alkyl or acyl group, contain at least one free amino group and at least one —COOH or —SO₃H group in the molecule and which are capable of forming inner salts. Examples of suitable ampholytic surfactants are N-alkyl glycines, N-alkyl propionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids containing around 8 to 18 carbon atoms in the alkyl group. Particularly preferred ampholytic surfactants are N-cocoalkyl amino-propionate, cocoacyl aminoethyl aminopropionate and C12-18 acyl sarcosine.

Nonionic surfactants contain, for example, a polyol group, a poly-alkylene glycol ether group or a combination of polyol and polyglycol ether groups as the hydrophilic group. Examples of such compounds are

products of the addition of 2 to 30 mol ethylene oxide and/or 0 to 5 mol propylene oxide onto linear fatty alcohols containing 8 to 22 carbon atoms, onto fatty acids containing 12 to 22 carbon atoms and onto alkylphenols containing 8 to 15 carbon atoms in the alkyl group,

C₁₂₋₂₂ fatty acid monoesters and diesters of products of the addition of 1 to 30 mol ethylene oxide onto glycerol,

C₈₋₂₂ alkyl mono- and oligoglycosides and ethoxylated analogs thereof,

products of the addition of 5 to 60 mol ethylene oxide onto castor oil and hydrogenated castor oil,

products of the addition of ethylene oxide onto sorbitan fatty acid esters and

products of the addition of ethylene oxide onto fatty acid alkanolamides.

Examples of cationic surfactants suitable for use in the formulations according to the invention are, in particular, quaternary ammonium compounds. Preferred quaternary ammonium compounds are ammonium halides, such as alkyl trimethyl ammonium chlorides, dialkyl dimethyl ammonium chlorides and trialkyl methyl ammonium chlorides, for example cetyl trimethyl ammonium chloride, stearyl trimethyl ammonium chloride, distearyl dimethyl ammonium chloride, lauryl dimethyl ammonium chloride, lauryl dimethyl benzyl ammonium chloride and tricetyl methyl ammonium chloride. Other cationic surfactants suitable for use in accordance with the invention are the quaternized protein hydrolyzates.

Also suitable for the purposes of the invention are cationic silicone oils such as, for example, the commercially available products Q2-7224 (manufacturer: Dow Corning; a stabilized trimethyl silyl amodimethicone), Dow Corning 929 Emulsion (containing a hydroxylamino-modified silicone which is also known as amodimethicone), SM-2059 (manufacturer: General Electric), SLM-55067 (manufacturer: Wacker) and Abil®-Quat 3270 and 3272 (manufacturer: Th. Goldschmidt; diquaternary polydimethyl siloxanes, Quaternium-80)

Alkyl amidoamines, particularly fatty acid amidoamines, such as the stearyl amidopropyl dimethyl amine obtainable as Tego Amid®S 18, are distinguished not only by their favorable conditioning effect, but also and in particular by their ready biodegradability. Quaternary ester compounds, so-called “esterquats”, such as the methyl hydroxyalkyl dialkoyloxyalkyl ammonium methosulfates marketed under the trade name of Stepantex® and the products marketed under the trade name of Dehyquart®, such as Dehyquart® AU-46, are also readily biodegradable.

One example of a quaternary sugar derivative suitable for use as a cationic surfactant is the commercially available product Glucquat®100 (INCI name: Lauryl Methyl Gluceth-10 Hydroxypropyl Dimonium Chloride).

The compounds containing alkyl groups used as surfactants may be single compounds. In general, however, these compounds are produced from native vegetable or animal raw materials so that mixtures with different alkyl chain lengths dependent upon the particular raw material are obtained.

The surfactants representing addition products of ethylene and/or propylene oxide with fatty alcohols or derivatives of these addition products may be both products with a “normal” homolog distribution and products with a narrow homolog distribution. Products with a “normal” homolog distribution are mixtures of homologs which are obtained in the reaction of fatty alcohol and alkylene oxide using alkali metals, alkali metal hydroxides or alkali metal alcoholates as catalysts. By contrast, narrow homolog distributions are obtained when, for example, hydrotalcites, alkaline earth metal salts of ether carboxylic acids, alkaline earth metal oxides, hydroxides or alcoholates are used as catalysts. The use of products with a narrow homolog distribution can be of advantage.

The preparations according to the invention preferably may also contain a conditioning agent selected from the group consisting of cationic surfactants, cationic polymers, alkyl amidoamines, paraffin oils, vegetable oils and synthetic oils.

Cationic polymers can be preferred conditioning agents. These are generally polymers containing a quaternary nitrogen atom, for example in the form of an ammonium group. The following are examples of preferred cationic polymers:

Quaternized cellulose derivatives commercially available under the names of Celquat® and Polymer JR®. The compounds Celquat® H 100, Celquat® L 200 and Polymer JR®400 are preferred quaternized cellulose derivatives.

Polymeric dimethyl diallyl ammonium salts and copolymers thereof with acrylic acid and with esters and amides of acrylic acid and methacrylic acid. The products commercially available under the names of Merquat®100 (poly(dimethyl diallyl ammonium chloride)), Merquat®550 (dimethyl diallyl ammonium chloride/acrylamide copolymer) and Merquat® 280 (dimethyl diallyl ammonium chloride/acrylic acid copolymer) are examples of such cationic polymers.

Copolymers of vinyl pyrrolidone with quaternized derivatives of dialkylaminoacrylate and methacrylate, such as vinyl pyrrolidone/dimethylaminomethacrylate copolymers quaternized, for example, with diethyl sulfate. Compounds such as these are commercially available under the names of Gafquat®734 and Gafquat®755.

Copolymers of vinyl pyrrolidone with methoimidazolinium chloride which are commercially available under the name of Luviquat®.

Quaternized polyvinyl alcohol.

The polymers with quaternary nitrogen atoms in the main polymer chain known by the names of Polyquaternium 2, Polyquaternium 17, Polyquaternium 18 and Polyquaternium 27.

Cationic polymers from the first four groups mentioned are particularly preferred, Polyquaternium 2, Polyquaternium 10 and Polyquaternium 22 being most particularly preferred.

Alternatively to the cationic polymers, zwitterionic or ampholytic polymers are particularly preferred conditioning agents. Preferred representatives are octyl acrylamide/methyl methacrylate/tert.butyl aminoethyl methacrylate/2-hydroxypropyl methacrylate copolymers and, in particular, acrylamidopropyl trimethylammonium chloride/acrylate copolymer.

Other suitable conditioning agents are silicone oils, more particularly dialkyl and alkylaryl siloxanes, such as for example dimethyl polysiloxane and methylphenyl polysiloxane, and alkoxylated and quaternized analogs thereof. Examples of such silicones are the products marketed by Dow Corning under the names of DC 190, DC 200, DC 344, DC 345 and DC 1401 and the products Q2-7224 (manufacturer: Dow Corning; a stabilized trimethyl silyl amodimethicone), Dow Corning® 929 Emulsion (containing a hydroxylamino-modified silicone which is also known as amodimethicone), SM-2059 (manufacturer: General Electric), SLM-55067 (manufacturer: Wacker) and Abil® Quat 3270 and 3272 (manufacturer: Th. Goldschmidt; diquaternary polydimethyl siloxanes, quaternium-80).

Other suitable conditioning agents are paraffin oils, synthetically produced oligomeric alkenes and vegetable oils, such as jojoba oil, sunflower oil, orange oil, almond oil, wheatgerm oil and peach kernel oil.

Phospholipids, for example soya lecithin, egg lecithin and kephalins, are also suitable hair-conditioning compounds.

Other active substances, auxiliaries and additives are, for example,

nonionic polymers such as, for example, vinyl pyrrolidone/vinyl acrylate copolymers, polyvinyl pyrrolidone and vinyl pyrrolidone/vinyl acetate copolymers and polysiloxanes,

anionic polymers such as, for example, polyacrylic acids, crosslinked polyacrylic acids, vinyl acetate/crotonic acid copolymers, vinyl pyrrolidone/vinyl acrylate copolymers, vinyl acetate/butyl maleate/isobornyl acrylate copolymers, methyl vinyl ether/maleic anhydride copolymers and acrylic acid/ethyl acrylate/N-tert.butyl acrylamide terpolymers,

thickeners, such as agar agar, guar gum, alginates, xanthan gum, gum arabic, karaya gum, locust bean gum, linseed gums, dextrans, cellulose derivatives, for example methyl cellulose, hydroxyalkyl cellulose and carboxymethyl cellulose, starch fractions and derivatives, such as amylose, amylopectin and dextrins, clays such as, for example, bentonite or fully synthetic hydrocolloids such as, for example, polyvinyl alcohol,

structurants, such as maleic acid and lactic acid,

perfume oils, dimethyl isosorbide and cyclodextrins,

solvents and solubilizers, such as ethanol, isopropanol, ethylene glycol, propylene glycol, glycerol and diethylene glycol,

quaternized amines, such as methyl-1-alkylamidoethyl-2-alkylimidazolinium methosulfate,

defoamers, such as silicones,

dyes for coloring the preparations,

antidandruff agents, such as piroctone olamine, zinc omadine and climbazol,

UV filters, more particularly derivatized benzophenones, cinnamic acid derivatives and triazines,

substances for adjusting the pH value, for example typical acids, more particularly food-grade acids and bases,

active substances, such as allantoin, pyrrolidone carboxylic acids and salts thereof and bisabolol,

vitamins, provitamins and vitamin precursors, more particularly those of groups A, B₃, B₅, C, E, F and H,

cholesterol,

consistency factors, such as sugar esters, polyol esters or polyol alkyl ethers,

fats and waxes, such as spermaceti, beeswax, montan wax and paraffins,

fatty acid alkanolamides,

complexing agents, such as EDTA, NTA, β-alanine diacetic acid and phosphonic acids,

swelling and penetration agents, such as glycerol, propylene glycol monoethyl ether, carbonates, hydrogen carbonates, guanidines, ureas and primary, secondary and tertiary phosphates,

opacifiers, such as latex, styrene/PVP and styrene/acrylamide copolymers,

pearlizers, such as ethylene glycol mono- and distearate and PEG-3-distearate,

pigments,

stabilizers for hydrogen peroxide and other oxidizing agents,

propellents, such as propane/butane mixtures, N₂O, dimethyl ether, CO₂ and air.

Information on other optional components and the quantities in which they are used can be found in the reference books known to the expert, for example Kh. Schrader, Grundlagen und Rezepturen der Kosmetika, 2nd Edition, Hüthig Buch Verlag, Heidelberg, 1989.

A third embodiment of the present invention is a process for blonding keratin fibers in which

if desired, a pretreatment preparation Ml is applied to the fibers,

a blonding preparation M2 containing at least one oxidizing agent is then applied to the fibers, another preparation M3 optionally being added to the preparation M2 immediately before application,

the blonding preparation M2 is rinsed off the fibers after a contact time of 5 to 30 minutes

and, after the treatment, a preparation M4 is optionally applied to the fibers and rinsed off again after a contact time of a few minutes,

at least one of the preparations M1, M2, M3 or M4 containing at least one compound corresponding to formula (I).

The preparation M1 is a pretreatment preparation containing at least one compound corresponding to formula (I) in a cosmetic carrier. Besides at least one oxidizing agent, the blonding preparation M2 may contain the ingredients mentioned above. A preparation M3 containing at least one compound corresponding to formula (I) is preferably added to the preparation M2 shortly before application. The resulting mixture corresponds to the preparation according to the invention. The preparation M3 containing at least one compound corresponding to formula (I) may be made up both as a solid and as an aqueous preparation.

According to the invention, the preparation M4 is a hair aftertreatment preparation as described, for example, in K. Schrader, Grundlagen und Rezepturen der Kosmetika, 2nd Edition, 1989, Dr. Alfred Hüthig Verlag, Heidelberg, pp. 722 et seq. which may optionally contain at least one compound corresponding to formula (I).

The following Examples are intended to illustrate the present invention.

### EXAMPLES

1) Preparation of the Blonding Creams

A reference blonding cream (cream 1) with no vitamin B6 (pyridoxine·HCl) and a blonding cream (cream 2) containing vitamin B6 were prepared (see Table 1).

TABLE 1
(all quantities in % by weight)

| | Cream 1 | Cream 2 (invention) |
|---|---|---|
| Hydrenol ® D¹ | 15% | 15% |
| Kokoslorol ® C12-18² | 2% | 2% |
| Eumulgin ® B2³ | 0.75% | 0.75% |
| Texapon ® NSO⁴ | 5% | 5% |
| Dehyton ® K⁵ | 3.75% | 3.75% |
| Pyridoxine · HCl | — | 2.0% |
| L-arginine | 1.0% | 1.0% |
| Gluadin ® W40⁶ | 1.0% | 1.0% |
| Sodium silicate | 0.2% | 0.2% |
| Ammonia⁷ | to pH 10 | to pH 10 |
| Water | to 100% | to 100% |

¹C₁₆₋₁₈ fatty alcohol (INCI name: Cetearyl Alcohol) (COGNIS)
²C₁₂₋₁₈ fatty alcohol (INCI name: Coconut Alcohol) (COGNIS)
³Cetylstearyl alcohol containing ca. 20 EO units (INCI name: Ceteareth-20) (COGNIS)
⁴Lauryl ether sulfate, sodium salt containing ca. 2 EO units (ca. 27.5% active substance, INCI name: Sodium Laureth Sulfate) (COGNIS)
⁵N,N-dimethyl-N-(C₈₋₁₈-cocoamidopropyl)-ammonium acetobetaine (ca. 30% active substance, INCI name: Aqua (Water), Cocamidopropyl Betaine (COGNIS)
⁶Wheat protein hydrolyzate (INCI name: Aqua (Water), Hydrolyzed Wheat Protein, Sodium Benzoate) Phenoxyethanol, Methylparaben, Propylparaben)
⁷25% aqueous solution

The substances Hydrenol®D, Kokoslorol®C12-18, Eumulgin®B2, Texpaon®NSO, Gluadin® W40 and Dehyton®K were melted at 80° C., mixed with the water heated to 80° C. and emulsified with vigorous stirring. The emulsion thus formed was then cooled with gentle stirring. The remaining components were added with stirring to the emulsion at a temperature of 40° C.

2). Demonstration of the Structuring Effect of Vitamin B6 when Applied Together with the Blonding Preparation

A) Analysis Method Used: HP-DSC (High-pressure Differential Scanning Calorimetry)

Thermoanalytical investigations are particularly suitable for characterizing two-phase systems to which human hair fibers as fibrous keratins with their crystalline α-helix component and amorphous matrix component also belong. On the one hand, glass transitions and aging behavior of the amorphous matrix can be investigated, on the other hand the melting behavior of the crystalline helical phase provides important information. Thermoanalytical studies were described for the first time in 1899. The first differential thermoanalyses (DTA) of protein fibers were carried out towards the end of the fifties (F. Schwenker, J. H. Dusenbury, Text. Res. J. 1963, 30, pages 800 et seq; W. D. Felix, M. A. McDowall, H. Eyring, ibid. (1963), 33, pages 465 et seq). In the following years, various thermoanalytical measuring techniques, such as DTA, HP-DTA (high-pressure DTA) and DSC (differential scanning calorimetry) were applied to keratin fibers, for example to investigate the phenomenon of supercontraction, α-β-phase transitions of the helices or denaturing processes. Recently, the method of IIP-DSC was used to study keratin fibers, more particularly at the Deutsches Wollforschungsinstitut (German Wool Research Institute) in Aachen (F. J. Wortmann, H. Deutz, J. Appl. Polym. Sci. 1993, 48, pp. 137 et seq.) IIP-DSC rules out the problems associated with pyrolytic effects which occur in conventional DSC and the problems with data acquisition and interpretation by which DTA is attended. DSC measurements are carried out on keratins which are encapsulated with water in commercially obtainable pressure-tight measuring capsules. In the keratin/water system, a high water vapor pressure from which the HP-DSC analysis derives builds up in the encapsulated steel crucibles on heating to >100° C. The crucial difference between the HP-DSC thermograms of human hair fibers and normal DSC thermograms is that the endothermal peaks which reproduce the transition point and the transition enthalpy are shifted by ca. 90° C. to lower temperatures. This derives from the fact that, after diffusing into the hair fibers, the water reduces protein stability by weakening and splitting hydrogen bridge bonds and salt bonds so that the “gluing temperature” of the keratins is reduced. If only hydrogen bridges and salt bridges are dissolved by the supercontracting agent, such as water, the thermal effect is reversible (supercontraction). However, the process becomes irreversible when covalent bonds, such as disulfide bridges for example, are split. This happens when human hair fibers are heated with water to >150° C. in pressure-tight capsules. The irreversible transition, interpreted as the transition of the α-helical regions in the proteins into a random state, results in endothermal peaks, the position of the peaks reproducing the transition point or even the denaturing point and their area reproducing the transition or denaturing enthalpy.

Accordingly, both structural and chemical states and changes in fiber keratins and particularly in human hair fibers can be detected by dynamic differential scanning calorimetry (DSC). Under precisely defined test conditions, the processes detectable by calorimetry in human hair fibers can be recorded in the form of thermograms and used in regard to peak positions, structures and areas as an indicator for influencing order/disorder transitions through changes in inner and/or outer parameters produced, for example, by cosmetic treatment of the hair fibers. In other words, information on the strength of or damage to human hair fibers can be obtained from the endothermal peaks recorded in the thermogram of human hair fibers on the basis of peak position (transition point) and peak area (transition enthalpy).

Detailed investigations into the influence of the cystine content on the denaturing of the α-helices in keratins have, for example, shown that the denaturing temperature (transition temperature) of the keratin increases linearly with the cystine content. The effect of the increased stability of the matrix region through the higher degree of crosslinking of the increased percentage of disulfide bridges in the matrix is that the transition of the helices embedded in this matrix is made difficult, resulting in an increase in the denaturing temperature. Conversely, a reduction in the denaturing temperature and above all in the denaturing enthalpy can generally be observed in human hair fibers treated by permanent waving or bleaching or coloring (H. Deutz, Doktorarbeit, RWTH Aachen 1993).

B) Procedure

Before application to a human hair tress (Klugmann, 6622D, medium blond), quantities of 50 ml of creams 1 and 2 were mixed with 40 ml of an aqueous H₂O₂ solution. In experiments 2.3 and 2.4, 10 g of a commercially available booster (96% by weight ammonium peroxodisulfate and 4% by weight silicon dioxide (amorphous) were added to the blonding formulation before application. All formulations have a mixed pH of 9.8 to 10.2. After a contact time of 30 minutes, the hair was rinsed and dried.

The denaturing temperatures of the blonded hair samples and the untreated hair were thermoanalytically determined by HP-DSC. In addition, lightening performance was determined by calorimetric analysis.

The results of the HP-DSC measurement and the calorimetric determination of the lightening effect of the respective formulations are set out in Table 2. The CIELAB co-ordinate L shown is a measure of the lightness and is calculated from the standard color values X, Y and Z which are in turn derived from the spectral distributions of the degree of reflection of the sample (H. G. Völz, Industrielle Farbprüfung, VCH, Weinheim, 1990). The higher the value for L, the better the lightening performance of the blonding preparation.

TABLE 2

| | | Denat. temp. | |
|---|---|---|---|
| Example | Mixture | [° C] | L |
| 2.0 | None (untreated hair) | 144.6 | 36.15 |
| 2.1 | Cream 1/H₂O₂ (6%) | 141.9 | 43.77 |
| 2.2 | Cream 2/H₂O₂ (6%) | 144.6 | 44.80 |
| 2.3 | Cream 1/H₂O₂ (12%)/10 g booster | 139.5 | 53.45 |
| 2.4 | Cream 2/H₂O₂ (12%)/10 g booster | 142.9 | 54.12 |

1. The use of compounds corresponding to formula (I): 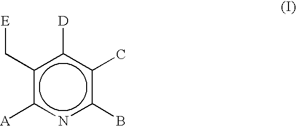
in which
A and B independently of one another represent hydrogen, halogen, a C₁₋₄ alkyl group, a C₃₋₆ cycloalkyl group, a C₁₋₄ monohydroxyalkyl group, a C₂₋₄ oligohydroxyalkyl group, a C₁₋₄ aminoalkyl group, a group —OR or a group —NR¹R², where R¹ and R² independently of one another represent hydrogen, a C₁₋₄ alkyl group or a C₁₋₄ monohydroxyalkyl group or R¹ and R² together with the nitrogen atom form a saturated ring,
C represents a group —OR, —NR¹R², —OP(O)(OR³)₂, a C₁₋₄ monohydroxyalkyl group, a C₂₋₄ oligohydroxyalkyl group or a C₁₋₄ alkyl group,
D represents a group —OR, a carboxy group, a C₁₋₂₂ alkoxycarbonyl group, a formyl group, a group —CH₂OR or a group —CH₂—NR₂,
E represents a group —OR, —OP(O)(OR³)₂, a C₁₋₄ monohydroxyalkyl group or a C₂₋₄ oligohydroxyalkyl group,
R representing hydrogen, a C₁₋₄ alkyl group, a C₁₋₂₂ acyl group, a hydroxy-C₂₋₂₂-acyl group, a C₂₋₁₀ carboxyacyl group, a C₃₋₁₀ oligocarboxyacyl group, an oligocarboxymonohydroxy-C₃₋₁₀-acyl group, an oligocarboxyoligohydroxy-C₃₋₁₀-acyl group, a C₃₋₈ cycloalkyl group, a C₁₋₄ monohydroxyalkyl group, a C₂₋₄ oligohydroxyalkyl group, an aryl group which may contain a hydroxy, nitro or amino group, a heteroaromatic group or a group —CH₂CH₂NR¹R², where R¹ and R² are as defined above,
R³ representing hydrogen or a C₁₋₅ alkyl group,
or one of the corresponding physiologically compatible salts, for reducing the damage to keratinous fibers, especially human hair, by oxidative processes.

2. The use claimed in claim 1, characterized in that one of the two groups A and B is hydrogen.

3. The use claimed in claim 1 or 2, characterized in that one of the two groups A and B is hydrogen and the other is a C₁₋₄ alkyl group.

4. The use claimed in any of claims 1 to 3, characterized in that C is a hydroxy group, a C₁₋₄ monohydroxyalkyl group or a C₂₋₄ oligohydroxyalkyl group.

5. The use claimed in any of claims 1 to 4, characterized in that D is a hydroxymethyl group, a group —CH₂—NR₂, a hydroxy group, a carboxy group or a formyl group.

6. The use claimed in any of claims 1 to 5, characterized in that E is a hydroxy group or a group —OP(O)(OH)₂.

7. The use claimed in any of claims 1 to 6, characterized in that the compound corresponding to formula (I) is selected from the group consisting of pyridoxine, pyridoxal, pyridoxamine or pyridoxal-5′-phosphate.

8. Preparations for the oxidative treatment of keratin fibers, more particularly for blonding hair, containing at least one oxidizing agent, characterized in that they contain at least one vitamin B6 derivative of formula (I) according to any of claims 1 to 7.

9. Preparations as claimed in claim 8, characterized in that they contain the compound corresponding to formula (I) in a quantity of 0.05 to 2% by weight, based on the preparation as a whole.

10. Preparations as claimed in claim 8 or 9, characterized in that they additionally contain a booster component.

11. A process for blonding keratinous fibers in which
if desired, a pretreatment preparation M1 is applied to the fibers,
a blonding preparation M2 containing at least one oxidizing agent is then applied to the fibers, another preparation M3 optionally being added to the preparation M2 immediately before application,
the blonding preparation M2 is rinsed off the fibers after a contact time of 5 to 30 minutes
and, after the treatment, a preparation M4 is optionally applied to the fibers and rinsed off again after a contact time of a few minutes,
characterized in that at least one of the preparations M1, M2, M3 or M4 contains at least one compound corresponding to formula (I).

12. A process as claimed in claim 11, characterized in that a preparation M3 containing at least one compound of formula (I) is added to the preparation M2 shortly before application.

13. A process as claimed in claim 11 or 12, characterized in that the preparation M2 is a preparation according to any of claims 8 to 10.
